(19) United States
(12) Patent Application Publication
(10) Pub. No.: US 2013/0344007 A1
(43) Pub. Date: Dec. 26, 2013

(54) FRANKINCENSE CHEWING GUM

(71) Applicants: D. Gary Young, Alpine, UT (US); Marc Schreuder, Provo, UT (US); Janelle Pack, Salt Lake City, UT (US)

(72) Inventors: D. Gary Young, Alpine, UT (US); Marc Schreuder, Provo, UT (US); Janelle Pack, Salt Lake City, UT (US)

(73) Assignee: YL HOLDINGS, INC., Lehi, UT (US)

(21) Appl. No.: 13/533,793

(22) Filed: Jun. 26, 2012

(51) Int. Cl. A61K 36/324; A61K 36/534; A61K 31/045; A61P 3/04; A61K 31/343; A61K 36/889; A61K 36/899; A61K 36/61; A61K 9/68; A61K 31/19

(52) U.S. Cl. 424/48; 424/725; 424/747; 424/769; 514/729; 514/557; 514/469; 424/727; 424/750; 424/742

(57) ABSTRACT

This disclosure relates to a composition that may include frankincense resin and various combinations of essential oils, which combinations may be effective in promoting weight loss. The present disclosure further relates to a weight loss and satiation composition that may include frankincense resin and a flavorant. In some embodiments, the composition may include a gum base.

### FIELD

The present disclosure relates to a chewing gum composition. Specifically, this disclosure relates to a composition with Frankincense along with essential oils and other compounds, the combination of which may be effective in promoting weight loss and feelings of satiation.

### BACKGROUND

In recent years, sales for essential oils and other topically applied oils have rapidly increased. Topically applied oils are usually oils which are derived from, or include certain natural essential components or essences of different substances from plants. Such topically applied oils are generally referred to as essential oils.

Essential oils are commonly known as nature's living energy, and as such are the natural, aromatic volatile liquids found in shrubs, flowers, trees, resins, fruit peels, rhizomes, roots, bushes, and seeds. The distinctive components in essential oils defend plants against insects, environmental. conditions, and disease. They are also vital for a plant to grow, live, evolve, and adapt to its surroundings. Essential oils are extracted from aromatic plant sources via steam distillation, cold pressing, and other types of distillation. Essential oils are highly onceritrated and far more potent than dry herbs. Other topically applied oils and fatty oils may include olive oil, almond oil, coconut oil, etc., and oils high in fats and esters, such as jojoba oil. and waxes such as beeswax.

While essential oils often have a pleasant aroma, their phytochemical makeup is complex and their benefits vast. Historically, essential oils have played a prominent role in everyday life. With more than 200 references to aromatics, incense, and ointments throughout the Bible, essential oils are said to be used for anointing and healing the sick. Today, essential oils are commonly used for aromatherapy, massage therapy, emotional health, personal care, nutritional supplements, household solutions, and much more. For example, essential oils have been used as active ingredients in various types of prescription, over-the-counter, and consumer products which are designed to provide various benefits. Essential oils have also been used as agents to improve the mental state of a user, thereby placing the user in a mental condition that is conducive to self-control and appetite control. Additionally, Frankincense has been known to have various beneficial effects on health and mental well-being.

While brain chemistry and mental conditioning are known to play a role in weight loss, there are many other physical, chemical and emotional factors that contribute to weight loss. For example, a large body of literature shows that many phytochemicals and natural products are used to treat obesity as metabolic stimulants, appetite suppressants, anti-inflammatories, and starch blockers and by regulation of glucose and lipid metabolism, as well as targeting adipocyte differentiation. Additionally, chewing gum has been used as a device to satiate the hand-to-mouth reflex as well as giving the mouth something to do to reduce snacking and overeating. Currently available weight loss compositions address some of these factors, but fail to address others. Thus, while compositions currently exist which are designed to promote weight loss, challenges still exist. Accordingly, it would be an improvement in the art to augment or even replace current techniques with other techniques and/or compositions.

### BRIEF SUMMARY

The present disclosure relates to a chewing gum composition for weight loss and satiation composition. Specifically, this disclosure relates to a composition that may include frankincense resin and various combinations of essential oils, which combinations may be effective in promoting weight loss. The present disclosure further relates to a weight loss and satiation composition that may include frankincense resin and a flavorant. In some embodiments, the composition may include a gum base.

The gum base may be between about 20 and 30% of the composition by weight and the Frankincense resin is between about 20 and 40% of the composition by weight. The composition may also include isomalt, and/or at least one essential oil, wherein the at least one essential oil may be selected from: peppermint, spearmint, and frankincense. The frankincense resin may be selected from Boswellia carteri, Boswellia sacra, Boswellia frereana, Boswellia papyrifera, Boswellia neglecta, and Boswellia serrata.

In some embodiments, the gum base may be between about 0 and 30% of the composition by weight and the Frankincense resin may be between about 10 and 90% of the composition by weight. The composition may also include isomalt, and/or at least one essential oil, wherein the at least one essential oil may be selected from: peppermint, spearmint, frankincense, coconut, eucalyptus, menthol, or any combination of these.

The frankincense resin may be selected from Boswellia carteri, Boswellia sacra, Boswellia frereana, and Boswellia serrata. The Frankincense resin may include Epi-lupeol, boswellic acid, incensole acetate, incensole, sesquiterpenes, diterpenes, triterpenes, tetraterpenes, any of their natural derivatives, or any combination of these. In some embodiments, the composition may also include at least one of stevia, calcium stearate, isomalt, xylitol, thaumatin, peppermint oil, spearmint oil, and a colorant. Similarly, the composition may include a sweetener, a flavor stabilizer, a texture stabilizer, xylitol, calcium stearate, or any combination of these.

### DETAILED DESCRIPTION

The present disclosure relates to a chewing gum composition that may be used as a weight loss and satiation aid. Throughout this specification, there are ranges defined by upper and lower boundaries. Each lower boundary can be combined with each upper boundary to define a range. The lower and upper boundaries should each be taken as a separate element.

In general, chewing gum compositions for weight loss and satiation may provide certain formulae and combinations of formulae which promote weight loss and other benefits. Some aspects may provide various nutraceutical compositions which may include various gum resins, essential oils, plants, herbs, extracts, spices, and pigments which have been found to be effective in promoting weight loss or other health-promoting effects. Thus, while specific examples of known compositions and combined nutraceutical therapies may be discussed, one having skill in the art will appreciate that the specific components may be substituted with natural or synthetic equivalents to achieve desired results within the spirit and teaching of chewing gum compositions for weight loss and satiation. Accordingly, the specific embodiments provided herein are not intended to be limiting, but rather are provided as a means for disclosing and teaching underlying principles.

Some embodiments may provide a gum composition for use in aiding weight loss and satiation comprising 1) Frankincense resin, and 2) an essential oil. In addition to the Frankincense resin and essential oil, the resulting product may include various combinations of active and inactive ingredients, wherein the final product promotes weight loss. The various components of the chewing gum products for use in weight loss and satiation are discussed below.

### Frankincense Resin

Frankincense has been used as a health and spiritual aid by many different societies throughout recorded history. Frankincense is produced by placing a cut in a Frankincense tree and then collecting the resin that leaks from the cut. There are forty-two different known species of Frankincense. Of these, six species are particularly well-known, Boswellia carteri, Boswellia sacra, Boswellia frereana, Boswellia papyrifera, Boswellia neglecta, and Boswellia serrata, and have been studied extensively to identify the various compounds in each variety. Considered the “holy anointing oil” in the Middle East, frankincense has been used in religious ceremonies for thousands of years. It was well known during the time of Christ and was one of the gifts given to Christ at his birth. Anciently, the Chinese used frankincense as a treatment for a range of ailments. Today, frankincense is still used worldwide for both its ceremonial and medicinal use. Frankincense can be used topically, as a dietary supplement, or diffused in the air for inhalation.

Some compounds in Frankincense known to provide various healthful effects include various terpene acids (such as aliphatic and aromatic terpenes, i.e., terpenes between C5-C40, including triterpene acids, such as boswellic acids, sesquiterpenes, diterpenes, triterpenes, tetraterpenes, and any of their natural derivatives, and incensole acetate), Epi-lupeol, and others. Boswellic acids have been shown to provide positive anti-inflammatory and immune system effects. Incensole and its natural derivatives has been shown to provide an anti-anxiety effect, anxiolytic improved mood, and nervous system protection. Epi-lupeol and its natural derivatives have been shown to provide an anti-inflammatory effect. Each of these positive effects may be effective in helping an individual control or lose weight. Each variety of Frankincense may have both various terpene acids and Epi-lupeol in various concentrations, or may not have one or the other at all. For example, Boswellia carteri and Boswellia sacra are both rich in boswellic acids while Boswellia frereana has no boswellic acids. Similarly, Boswellia frereana is rich in Epi-lupeol. Research into the various Frankincense varieties continues and other beneficial compounds are sure to be identified, particularly with high molecular weight compounds that are extracted in low quantities during distillation of essential oils such as boswellic acids, Epi-lupeol, and incensole.

Frankincense resin may also contain health stimulating compounds not commonly present in high concentrations in distilled frankincense essential oil. For example Boswellic acids, incensole, and Epi-lupeol have all been found in low concentrations in distilled frankincense essential oils. However, these compounds and their natural derivatives are found in higher abundance in the natural frankincense resin. Therefore, chewing the natural resin, such as in a chewing gum, may introduce higher concentrations of high molecular weight diterpenes, triperpenes, tetraterpenes, and their natural derivatives to the body during the mastication process.

Mood and anxiety may have a large impact on eating habits and diet. People who exhibit high-stress levels have been shown to have different eating habits than people who are better able to control their stress levels. People who feel good tend to make better decisions for themselves and for others. People who are happy tend to exercise more and make better dietary choices. Similarly, inadequate sleep due to stress or other factors may also lead to worse food choices. Also, healthy people with strong immune systems are also generally better able to make good food choices.

Inflammation has been shown to affect a variety of different systems in the body, including weight gain and weight retention. The diet we eat greatly influences our inflammatory response. Without inflammation, wounds and infections would never heal, but several diseases have been linked to chronic inflammation—the over abundance of cytokines in the blood can cause cascading effects in the onset of Type 2 diabetes. Chronic inflammation has been linked to several other diseases including cancer, heart disease, atherosclerosis, rheumatoid arthritis and Alzheimer's. Inflammation requires a lot of energy from the body, and puts it in a state of alert. Premature aging, anxiety, and stress and come from lack of energy and balance in the bloods immune system.

Because Frankincense is a resin, it may be used in chewing gums as a gum base or along with another gum base to provide the characteristics of chewing gum. Some varieties of Frankincense have been shown to maintain masticability for extended periods of time, which may provide a sort-of time release of the beneficial compounds of Frankincense described along with being an adequate chewing gum base. Frankincense resin may be powdered, crushed, or partially liquefied. For example, Frankincense resins may be included in a chewing gum in amounts between about 10% to about 99% by total weight (BTW).

In another example, a first gum base (such as those commonly used in making chewing gums) may be included in amounts between about 10% to about 95% BTW, and Frankincense resin may be included in amounts between about 10% to about 95% BTW. In another example, a first gum base may be included in amounts between about 20% to about 40% BTW, and Frankincense resin may be included in amounts between about 20% to about 40% BTW. In some embodiments, other gum bases may be used along with the Frankincense resin, such as acacia resin, also known as Arabic gum, chicle resin, or any other suitable natural or artificial gum compounds. Such other gums may be used in place of the gum base in whole or in part.

### Other Natural Active Ingredients

Any suitable essential oil can be employed in embodiments of a weight loss and satiation composition provided (1) the essential oil has therapeutic properties (e.g., the essential oil is effective in promoting weight loss and satiation), (2) the essential oil remains thermally stable in the composition, and (3) the essential oil is non-toxic to mammals (e.g., humans) and will be suitable for oral administration. Additionally, essential oils may be used to provide a primary flavor to the chewing gum, or may be used to mask or modify the natural flavor of the Frankincense resin. Preferably, the thermostability of the essential oil is over a prolonged period of time, e.g., up to about 3 years, up to about 1 year, or up to about 6 months, typically experienced in the manufacturing, packaging, shipping, and/or storage of the composition. The preferred essential oil will also preferably comply with any controlling or governing body of law.

Suitable specific essential oils may be derived from and include one or more of the following: ajowan, sweet almond, allspice, aloe vera, ammi visnaga (khella), amyris, angelica root, angelica seed, anise, anise seed, star anise, apricot kernel, absolute arnica, avocado, unrefined avocado, Copaiba balsam, balsam Peru genuine, balsam Peru, balsam peru liquid resin, balsam tolu, sweet french basil, basil, basil ct. methyl chavicol, lemon ct. citral basil, sweet ct. linalool basil, bay laurel, bay leaf, bay rum, bay leaf West Indies, bees wax, unrefined bees wax, benzoin absolute, benzoin resinoid, bergamot, mint bergamot, Italian bergamot, free bergaptene bergamot, birch, sweet birch, borage oil, boronia, butter, buchu leaf, cajeput, calamus, calendula oil, infused calendula oil, camellia oil, cannabis, caraway, caraway seed, cardamom, absolute carnation, carrot seed, high carotol carrot seed, carrot seed oil, cassia, cassis bud (black currant), castor oil, catnip, oil of catnip, cedarleaf, western red cedarleaf, cedarwood, Atlas cedarwood, Himalayan cedarwood, Virginia cedarwood, celery seed, chamomile, blue chamomile, German chamomile, Moroccan chamomile, Moroccan wild chamomile, Roman chamomile, champaca, cilantro, true cinnamon bark, cinnamon bark, cinnamon leaf, cinnamon cassia, cistus, citronella, Java citronella, ciste oil, artificial civet, clary sage, high sclareol clary sage, clementine, Italian clementine peel, clove, clove bud, clove leaf, cocoa, cocoa butter, unrefined cocoa butter, coconut, refined coconut, cognac, combava petitgrain, coriander, green coriander, cornmint, costus, cumin, cypress, davana, dill, dill weed, elemi, erigeron (fleabane), eucalyptus citriodora, eucalyptus globulus, lemon eucalyptus, fennel, sweet fennel, fenugreek, fir (i.e. abies ssp.), fir needles (i.e. Canada fir needle, Siberia fir needle, white fir needle, etc.), frankincense, India frankincense, Oman frankincense, galbanum oil, garlic, genet, geranium, geranium leaf, geranium rose, Bourbon geranium, Egyptian geranium, ginger, Cochin extra ginger, ginsing, Siberian ginsing, Korean ginsing, grapefruit, pink grapefruit, white grapefruit, grapeseed, hazelnut, helichrysum, helichrysum immortelle, Mad. helichrysum, Balkan helichrysum, Corsica helichrysum, France helichrysum, hemp oil, absolute honeysuckle, hyssop, hyssop decumbens, absolute immortelle, fragrant aster inula, Jamaican gold, unrefined Jamaican gold, jasmine, absolute jasmine, grandiflorum jasmine, sambac jasmine, jojoba oil, helio-carrot in jojoba, melissa in jojoba, absolute jonquille, juniper (i.e. juniperus ssp), juniper berry, (i.e, Siberia juniper berry, Croatia juniper berry, etc.) lanolin, unrefined anhydrous lanolin, lantana camara, laurel nobilis, lavandin, abrialis lavandin, grosso lavandin, lavender, Oregon lavender, Bulgarian lavender, Russian lavender, high-altitude lavendar, wild-crafted lavender, lavendin, organic lavindin, lemon, lemongrass, lime, distilled lime, expressed lime, litsea, litsea cubeba, blue, pink and white lotus, macadamia oil, mace, green mandarin, red mandarin, yellow mandarin, manuka, absolute marigold, marigold flower, marjoram, Spanish marjoram, sweet marjoram (true), massoia bark, melissa, codistilled melissa, “rectified” melissa, true melissa, absolute mimosa, mimosa, monarda, mugwort, musk seed, myrrh, myrtle, absolute narcissus, neroli (orange blossom), niaouli, nutmeg, extra nutmeg, oakmoss, absolute oak moss, ocotea, olibanum, absolute opopanax, bitter orange, blood orange, sweet orange, wild West Indian orange, oregano, orris root, concrete orris, osmanthus, palm, refined palm, palmarosa, paprika, parsley seed, patchouli, Indian patchouli oil, Indonesian patchouli, peanut, pecan, pennyroyal, pepper, black pepper, super black pepper, peppermint, India peppermint, USA baby mint peppermint, petitgrain (orange leaves), alcoquiana, pine (i.e. pinus ssp), pine needle (i.e., white pine needle, etc.), evening primrose, ravensara anisata, true ravensara, ravensare, ravintsara, redberry, rosalina, rose, rose geranium, rose otto, Bulgarian rose, English rose, Turkish rose, rosehip seed oil, rosemary, rosemary anti-oxidant extract powder, rosemary verbenone, Morocco rosemary, Spain rosemary, rosewood, rosewood oil, rue, sage, white sage, sage dalmatian, sage officinalis, sage triloba, sandalwood, seabuckthorn berry, sesame oil, sesame seed oil, shea butter, unrefined shea butter, spikenard, green spikenard, spruce (i.e., picea ssp.), St. John's wort, styrax resin, tagetes, tangerine, Dancy tangerine, tarragon, tea tree, Australia tea tree, thuja (cedar leaf), thyme, red thyme, thyme ct. linalool, thyme vulgaris, wild thyme, red thyme, mixed tocopherols, tolu balsam resin, absolute tuberose, tuberose, tumeric, valerian, vanilla, pure vanilla extract, vanilla bean, absolute vanilla bourbon, vegetable glycerin, absolute verbena, vetiver, violete leaves, vitex, organic Haiti vetiver, absolute violet leaf, walnut oil, wintergreen, natural wintergreen, wormwood, yarrow, ylang ylang, ylang ylang I, ylang ylang II, ylang ylang III, ylang ylang compound, ylang ylang complete, and ylang ylang extra. Specifically, suitable exemplary essential oils may include citral, cinnamon cassia, ocotea, pomegranate seed oil, or a combination thereof.

Other essential oils and additives may include, laurus nobilis, melaleuca alternifolia, m quienquenervia, m ericifolia, hibiscus abelmoschus, beeswax absolute (apis mellifera), bergamot mint (mentha citrate), bougoin de cassis (ribes nigrum), broom absolute (spartium junceum), cajuput (melaleuca leucadendron), camphor (cinnamomum camphora), cardamon seed (ellettaria cardamomum), cassie absolute (acacia farnesiana), everlasting absolute (helichrysum stoechas), hay absolute, ho wood (cinnamomum camphora), labdanum absolute (cistus ladaniferus), lentisque absolute (pistacia lentiscus), orange flower absolute (citrus aurantium), origanum, savory (satureja montana), spearmint (mentha spicata), tobacco absolute (spp. nicotiana), tonka absolute (dipteryx odorata), treemoss absolute (evernia prunastri), vetyvert (vetivera zizanoides), d limonene, 1 Limonene, mycrene, cineole, menthol, menthone, menthyl acetate, a humulene, gamma humulene, caryophyllene, borneol, linalool, linalyl acetate, methyl salicylate, bornyl acetate, para cymene, eugenal, geraniol, nerol, citronellol, sabinene, alpha pinene, beta pinene, eudesmol, aromadendrebe, globuluol, pterpinen 4 ol, terpinolene, cuminal aldehyde, alpha terpineol, bisaboilol, bisabolol oxides, ocimene, myrcene, fenchol, germacrene D, C, B, a zingiberene, gamma cadinene, beta selinene, farnesol, delta cadinene, alpha selinene, beta selinene, gamma selinene, delta elemene, alpha elemene, piperine, carvone, benzaldehyde, anisyl acetate, anisyl alchohol, camphene, geranyl acetate, isomenthol, isomenthone, vanillin, terpineol, valencene, sinensal, nootketone, gamma terpinene, thymol, carvacrol, a bergamotene, cubebol, merolidol, neryl acetate, methyl eugenol, longifolene, anethole, anisyl acetate, benzyl benzoate, benzyl cinnamate, 5-epiprezizane, khusimene, a-muurolene, khusimone, calacorene, b-humulene, a-longipinene, d-cadinene, valencene, calarene-gurjunene, a-amorphene, epizizanal, 3-epizizanol, khusimol, Isoeugenol, a curcumene, b curcumene, gamma curcumene, perryl alcohol, Iso-khusimol, Valerenol, b-vetivone, a-vetivone, dfN-Butyl-2-Methylbutyrate, Ethyl-2-Methylbutyrate, Isoamyl Isovalerate, Isopropyl-2-Methylbutyrate, 2-Methylbutyric Acid, Methyl-2-Methylbutyrate, Methyl Isovalerate, Phenylethyl Isovalerate, Benzyl Butyrate, Benzyl Isobutyrate, Benzyl Isovalerate, Benzyl Propionate, Butyl Butyrate, Ethyl Isovalerate, Ethyl Valerate, N-Hexyl Isobutyrate, N-Hexyl-2-Methylbutyrate, Isoamyl-2-Methylbutyrate, Isobutyl-2-Methylbutyrate, Phenethyl Butyrate, Phenethyl Isobutyrate, Phenethyl-2-Methylbutyrate, or any combination thereof.

The amount of essential oils (or other compounds) used in the chewing gum may be within the effective ranges of the individual oils. For example, in one embodiment a single essential oil may be added to the system within an effective range of 0.001 to about 2% BTW.

In another embodiment, a first essential oil may be added to the system within an effective range of 0.01 to 0.1% BTW, and a second essential oil may be added to the system within an effective range of 0.3 to 1.9% BTW. In another embodiment, a first essential oils may be added to the system within an effective range of 0.01 to 0.1% BTW, a second essential oil may be added to the system within an effective range of 0.01 to 0.1% BTW, and a third essential oil added to the system within an effective range of 0.2 to 1.1% BTW. In other embodiments, various combinations of oils may be added in various quantities which provide to maintain masticability and form while also providing therapeutic benefits.

Exemplary chewing gum compositions for weight loss can be produced by a variety of methods. In some embodiments, an effective amount of each of the components described above may be preformulated in separate solutions. Thereafter, the components may be mixed in effective ratios, as discussed above, along with other non-active ingredients. Any optionally added ingredients, such as colorants, flavorings, sweeteners, dietary supplements, fruits, flavor and structure stabilizers, gum bases, rheology modifiers, emulsifiers, colorants, and the like can be preferably added as desired. For example, isomalt, xylitol, malitol, stevia, sugars, natural flavors, natural sweeteners, Thaumatin, Calcium stearate, etc., may be added in proportions as desired to achieve a desired chewing gum product.

### EXAMPLES

### Example 1

Some embodiments may include Frankincense resin, isomalt, stevia, blueberry juice concentrate, Pure Fruit sweetener, peppermint essential oil, spearmint essential oil, and thaumatin.

### Example 2

Some embodiments may include a gum base, Frankincense resin, stevia, peppermint essential oil, colorant, spearmint essential oil, and xylitol.

### Example 3

Some embodiments may include a gum base, Frankincense resin, isomalt, stevia, peppermint essential oil, spearmint essential oil, and calcium stearate.

### Example 4

Some embodiments may include a gum base, Frankincense resin, isomalt, stevia, coconut essential oil, banana essential oil, malitol, and natural fruit flavors.

### Example 5

Some embodiments may include a gum base, Frankincense essential oil, stevia, peppermint essential oil, spearmint essential oil, and xylitol.

### Example 6

Some embodiments may include a chewing gum which includes any combination of the unique ingredients disclosed above in Examples, 1-5.

Other specific forms and combination of weight loss systems may be performed and provided without departing from its structures, methods, or other essential characteristics as broadly described herein and claimed hereinafter. The described embodiments are to be considered in all respects only as illustrative, and not restrictive. The scope is, therefore, indicated by the appended claims, rather than by the foregoing description. All changes that come within the meaning and range of equivalency of the claims are to be embraced within their scope.

What is claimed is:

1. A chewing gum composition for promoting weight loss, comprising:
Frankincense resin; and
a flavorant.

2. The composition of claim 1, further comprising, gum base.

3. The composition of claim 2, wherein the gum base is between about 20 and 30% of the composition by weight and the Frankincense resin is between about 20 and 40% of the composition by weight.

4. The composition of claim 1, wherein the Frankincense resin is between about 10 and 95% of the composition by weight.

5. The composition of claim 1, further comprising, isomalt.

6. The composition of claim 1, further comprising at least one essential oil.

7. The composition of claim 6, wherein the at least one essential oil is selected from: peppermint, spearmint, and frankincense.

8. The composition of claim 1, wherein the Frankincense resin is selected from Boswellia carteri, Boswellia sacra, Boswellia frereana, Boswellia papyrifera, Boswellia neglecta, and Boswellia serrata.

9. The composition of claim 8, wherein the Frankincense resin includes Epi-lupeol, or a natural derivative of Epi-lupeol.

10. The composition of claim 8, wherein the Frankincense resin includes boswellic acid, or a natural derivative of a boswellic acid.

11. The composition of claim 8, wherein the Frankincense resin includes incensole, or a natural derivative of incensole.

12. The composition of claim 1, further comprising at least one of: stevia, calcium stearate, isomalt, xylitol, theumatin, peppermint oil, spearmint oil, and a colorant.

13. The composition of claim 1, further comprising a sweetener.

14. The composition of claim 1, further comprising a flavor stabilizer.

15. The composition of claim 1, further comprising a texture stabilizer.

16. The composition of claim 1, further comprising xylitol.

17. A weight loss and satiation system, comprising:
a gum base;
frankincense resin;
a sweetener;
a colorant;
a natural flavorant; and
an essential oil.

18. The weight loss system of claim 17, wherein the sweetener is one of a plurality of sweeteners, and wherein the plurality of sweeteners includes at least stevia, isomalt, and xylitol.

19. The weight loss system of claim 17, further comprising calcium stearate and isomalt.

20. The weight loss system of claim 17, wherein the essential oil is one of a plurality of essential oils, and wherein the plurality of essential oils are selected from the group:
peppermint oil;
spearmint oil;
coconut oil;
lemon grass oil;
frankincense oil;
menthol oil; and
eucalyptus oil.
